(19) United States
(12) Patent Application Publication
(10) Pub. No.: US 2007/0054316 A1
(43) Pub. Date: Mar. 8, 2007

(54) Radical activated cleavage of biologics and microfluidic devices using the same

(76) Inventor: Robert J. Wiener, Middletown (DE)

Correspondence Address: DAY PITNEY LLP, 7 TIMES SQUARE, NEW YORK, NY, 10036-7311, US

(21) Appl. No.: 10/548,925

(22) PCT Filed: Jan. 13, 2004
(86) PCT No.: PCT/US04/00734

§ 371 Date: Sep. 12, 2005

Related U.S. Application Data
(63) Continuation of application No. 10/346,208, filed on Jan. 16, 2003, now Pat. No. 6,935,053.

(51) Int. Cl. G01N 33/53; C12Q 1/68; C12M 3/00

(52) U.S. Cl. 435/7.1; 435/4; 435/6; 422/186.04; 204/165; 435/297.2

(57) ABSTRACT

Disclosed is a cleavage method for biological sample characterization using hydroxyl radical activated cleavage in place of traditional enzymatic approaches. The hydroxyl radicals are generated from a semiconductor excited by an energy source. A microfluidic device for the two-dimensional separation of biological samples by hydroxyl radical activated cleavage is also disclosed.

### FIELD OF THE INVENTION

The present invention relates to methods of cleaving biomolecular components in biological samples using radical activated cleavage, and more particularly to the cleavage of biomolecular components in a microfluidic device with the use of hydroxyl radicals.

### BACKGROUND OF THE INVENTION

Cleavage of biomolecular components in a biological sample and subsequent pattern recognition of the fragments are the major paradigms for identification of the biomolecular components in the sample. Typically, an enzyme digest (e.g., a trypsin digest) is used for cleavage of biomolecular components containing amino acid sequences (e.g., proteins). Tryptic digest is extensively used as it provides highly specific cleavage at arginine and lysine residues. However, proteolytic enzymes require careful storage and preparation, and the use of the enzymes is often time consuming and labor intensive because the enzymes do not remain active over large temperature differentials and pH. In addition, proteolytic enzymes introduce noise into the detection system. For example, because proteolytic enzymes are proteins, self-digestion produces fragments that are detected but are not components of the sample protein. This can significantly affect detection limits of these biological samples. This is true even when the digest is performed with the use of a microfluidic device.

In view of the art, there is a need for a method for cleaving biomolecular components in a biological sample that avoids the time-consuming and labor intensive cleaving protocols associated with enzymatic digestion. Accordingly, it is an object of the present invention to provide a more effective and reliable method of cleaving biomolecular components in a biological sample.

### SUMMARY OF THE INVENTION

The present invention provides a method for cleaving biomolecular components in biological samples using hydroxyl radicals generated from semiconductors excited by an energy source. Biological samples include any liquid sample with a biomolecular component having an oxygen-containing backbone. Examples of biomolecular components to be cleaved in a biological sample include, but are not limited to, amino acid sequences, nucleic acid sequences, polysaccharides, and combinations thereof. The semiconductors to be used to generate the hydroxyl radicals in water containing environments are any inorganic semiconductor. Representative examples of inorganic semiconductors include, but are not limited to, titanium dioxide, zinc oxide, and combinations thereof. Examples of energy sources for exciting the energy source are light energy, thermal energy, and electrical energy. In a more preferred embodiment, light energy is used preferably in the ultraviolet bandwidth.

The present invention also provides a microfluidic device for cleaving the biomolecular components using the hydroxyl radicals. In a preferred embodiment, the microfluidic device includes: a channel including an inorganic semiconductor disposed on a portion of its interior surface, an energy source disposed at a position external to the channel, separation channels, and detector means.

Advantageously, the method of the present invention overcomes the problems with the current technology of separating and identifying biological samples, which requires, most often, the use of protease digestion of separated components and complicated interfaces to effect this digestion. Radical activated cleavage using semiconductors can be used in a variety of environments, does not require special storage or preparation, and can be regenerated infinitely. The cleavage process is highly tunable as the reaction can be terminated by removing the biological sample from contact with the semiconductor or by turning off the excitation source. Removal of the sample from the covalently bonded semiconductor removes any reactive species from the sample solution and terminates the cleavage mechanism. Using this process, a method of sample cleavage that is tunable through reaction time and radical production is possible. Another particular advantage of the present invention is that production of hydroxyl radicals using semiconductors avoids possible contamination that may occur with protease digestion. In addition, providing the device with a semiconductor for cleavage does not require complex experimental protocols as compared to the immobilization of proteolytic enzymes in a microfluidic device. These and other advantages of the invention will become more readily apparent from the detailed description set forth below.

### BRIEF DESCRIPTION OF THE DRAWINGS

FIG. 1 is a diagrammatic representation of a microfluidic device to be used in accordance with the invention.

FIG. 2 is a graph of absorbance units versus wavelength for 2-hydroxyterephthalic acid produced through hydroxyl radical generation from titanium dioxide in accordance with the invention.

FIG. 3A is an electropherogram of absorbance units versus time for 3 mg/mL of myoglobin diluted to 0.3 mg/mL with N-Tris(hydroxymethyl)methyl-3-aminopropanesulfonic acid prior to exposure to illuminated titanium dioxide in accordance with the invention.

FIG. 3B is an electropherogram of absorbance units versus time for 3 mg/mL of myoglobin diluted to 0.3 mg/mL with N-Tris(hydroxymethyl)methyl-3-aminopropanesulfonic after having been exposed to illuminated titanium dioxide for 1.75 hours in accordance with the invention.

FIG. 4A is an electropherogram of absorbance units versus time for 3 mg/mL of myoglobin diluted to 0.3 mg/mL with N-Tris(hydroxymethyl)methyl-3-aminopropanesulfonic after having been exposed to illuminated titanium dioxide for 2 hours in accordance with the invention.

FIG. 4B is the electropherogram of FIG. 4A showing a smaller scale of absorbance.

FIG. 5 is an electropherogram of absorbance units versus time for 2 mg/mL myoglobin diluted to 0.2 mg/mL with N-Tris(hydroxymethyl)methyl-3-aminopropanesulfonic acid prior to exposure to illuminated titanium dioxide in accordance with the invention.

FIG. 6A is an electropherogram of absorbance units versus time for 2 mg/mL of myoglobin diluted to 0.2 mg/mL with N-Tris(hydroxymethyl)methyl-3-aminopropanesulfonic acid after having been exposed to illuminated titanium dioxide for 20 minutes in accordance with the invention.

FIG. 6B is the electropherogram of FIG. 6A showing a smaller scale of absorbance.

FIG. 7A is an electropherogram of absorbance units versus time for 2 mg/mL of myoglobin diluted to 0.2 mg/mL with N-Tris(hydroxymethyl)methyl-3-aminopropanesulfonic acid after having been exposed to illuminated titanium dioxide for 30 minutes in accordance with the invention.

FIG. 7B is the electropherogram of FIG. 7A showing a smaller scale of absorbance.

FIG. 8A is an electropherogram of absorbance units versus time for 2 mg/mL of myoglobin diluted to 0.2 mg/mL with N-Tris(hydroxymethyl)methyl-3-aminopropanesulfonic acid after having been exposed to illuminated titanium dioxide for 45 minutes in accordance with the invention.

FIG. 8B is the electropherogram of FIG. 8A showing a smaller scale of absorbance.

### DETAILED DESCRIPTION OF THE INVENTION

The present invention provides a method for cleaving biomolecular components in biological samples through the use of hydroxyl radicals produced by excited inorganic semiconductors. The cleavage method of the invention advantageously avoids the time consuming and labor intensive protocols of enzyme digests while at the same time providing highly specific cleavage.

Biological samples to be used in accordance with the present invention are any liquid sample with a biomolecular component having an oxygen-containing backbone (e.g., amide linkages, phosphodiester linkages, and glycosidic linkages (i.e., ether)). Representative examples of biomolecular components to be cleaved in a biological sample include, but are not limited to, amino acid sequences, nucleic acid sequences, polysaccharides, and combinations thereof.

Semiconductors to be used to generate the hydroxyl radicals in accordance with the invention are any inorganic semiconductor. In a particularly preferred embodiment, the method of the present invention omits the use of an organic cleavage agent (e.g., a proteolytic enzyme). While wishing not to be limited by theory, as known in the art, semiconductors have an energy differential between their valence bands and their conducting bands. When energy impinges on the semiconductor, an electron is excited from the valence band to the conduction band. The excitation process creates an electronic charge carrier in the conduction band resulting in the ejection of an electron and an electron vacancy (i.e., a hole) in the valence band. When the highly oxidizing electron and hole produced from this phenomenon migrate to the surface, an oxidation reaction can occur. Frequently and in the presence of water dissociated on the surface, this reaction results in the production of hydroxyl radicals.

Examples of inorganic semiconductors that produce hydroxyl radicals in water containing environments include, but are not limited to, titanium dioxide, zinc oxide, and combinations thereof. In a preferred embodiment of the present invention, titanium dioxide is used as a photocatalyst for generating hydroxyl radicals. Light absorption in titanium dioxide is preferably accomplished using a wavelength in the range of 250 to 900 nm, with 350 to 450 nm being more preferred, in order to effect the promotion of an electron from the valence band to the conduction band of the semiconductor. The titanium dioxide is preferably illuminated by ultraviolet light. For example, because the valence band edge of titanium dioxide occurs at approximately +3.2 eV versus the normal hydrogen electrode (at pH 0) the hole is a very powerful oxidizing agent and is capable of generating hydroxyl radicals in water.

While wishing not to be limited by theory, the coating process of the semiconductor is performed following conventional techniques known in the art, such as adsorptive modification through Van der Waals force, hydrogen bonding or electrostatic interaction, direct covalent modification through a silane bond, and indirect covalent modification through a silane or polymer linker. For example, the semiconductor can be deposited on the interior surface of a substrate followed by annealing or applied to a colloid of solid particles constrained in a channel of a microfluidic device. In a preferred embodiment of the present invention, the inorganic semiconductor is immobilized in a microfluidic device by a covalently bonding it to the interior surface of one of the channels.

In accordance with the present invention, hydroxyl radicals are formed by exciting the inorganic semiconductor with an energy source in the presence of an aqueous medium. In the context of the invention, an aqueous medium is defined as water or a mixture of a majority of water and preferably one or more water-miscible organic solvents. Examples of energy sources suitable for exciting the semiconductor material thereby bridging the band gap energy include, but are not limited to, light energy, thermal energy, and electrical energy. In a more preferred embodiment, light energy is used preferably in the ultraviolet bandwidth. The energy source while external to the channel can be located on the chip (i.e., internally) or located external to the chip in the form of a lamp or fiber optic.

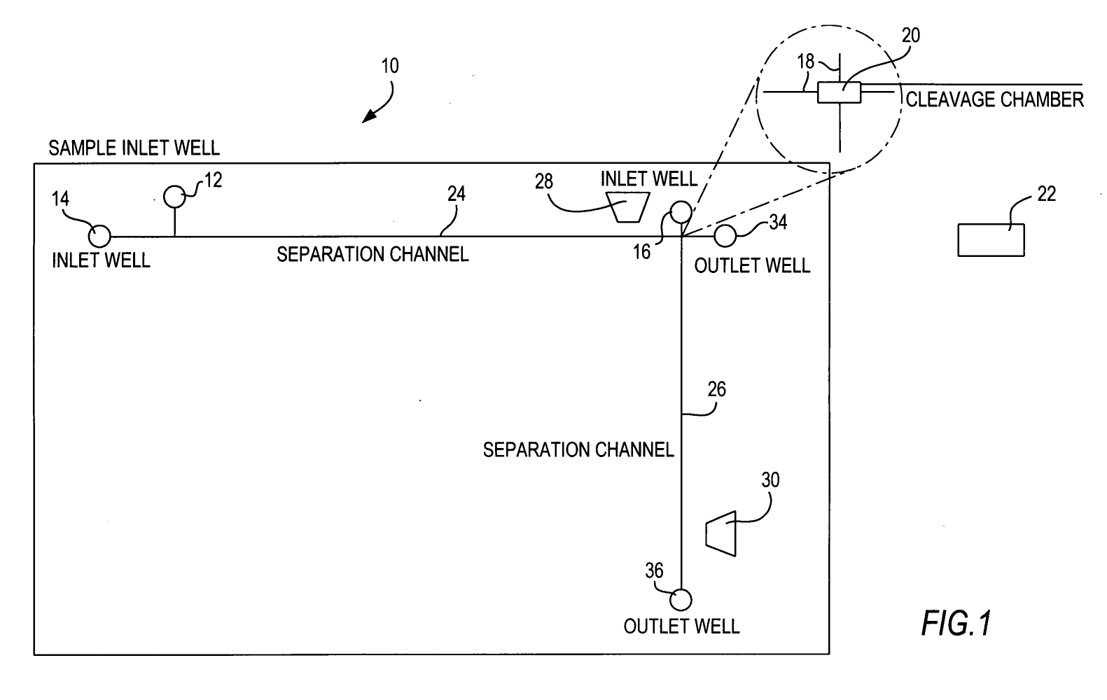

In an additional embodiment of the present invention, a microfluidic device is provided for cleaving biomolecular components in accordance with the invention. Referring to FIG. 1, a microchip 10 having a plurality of channels is illustrated. The channels are constructed out of any suitable material known in the art, such as fused silica, polydimethylsiloxane, polycarbonate, and combinations thereof. As shown in FIG. 1, the microchip 10 includes a sample inlet well 12 and aqueous medium inlet wells 14 and 16. A channel 18 includes an inorganic semiconductor disposed on a portion of its interior surface. The portion of the channel containing the inorganic semiconductor is in the form of a cleavage chamber 20. An energy source 22 is disposed at a position external to the channel 18. Separation channels 24 and 26 are located at a position intermediate the sample inlet well 12 and the channel 18 including the inorganic semiconductor. Detector means 28 and 30 are included. Microchip 10 is electronically coupled to a power source 32. Outlet wells 34 and 36 are fluidly coupled to the plurality of channels at a position distal from the sample inlet well 12.

In a further embodiment of the present invention, a method of cleaving a biomolecular component in a biological sample in a microfluidic device is provided in accordance with the invention. Returning to FIG. 1, a biological sample is introduced into the sample inlet well 12 of microchip 10 electrokinetically by applying a potential across the microchip 10. An aqueous medium (e.g., a buffer) is provided in inlet wells 14 and 16. The biological sample flows into separation channel 24 where it is separated according to electrophoretic mobility. The biological sample is passed to detector means 28 via electroosmotic flow. Channel 18 of the microchip 10 including an inorganic semiconductor is excited by the energy source 22 to produce hydroxyl radicals due to contact of the aqueous medium with inorganic semiconductor in cleavage chamber 22. The cleaved biomolecular component is detected at detector means 30. Detection is measured by either ultraviolet adsorption or fluorescence. Waste egresses from the microchip at outlet wells 34 and 36.

The invention will be better understood from, but is not limited to the Examples below. The reagents and materials referred to in the Examples are as follows. Titanium tetraisopropanate (Tyzor TPT) was provided by DuPont Chemicals. House skeletal myoglobin, ribonuclease-A, and lysozyme were obtained from Sigma-Aldrich (St. Louis, Mo.). Poly(hydroxyethy)acrylic acid (PHEA) was provided in a 10% (w/v) experimental solution from Cambrex and then diluted with deionized water. Polydimethlysiloxane (PDMS) was obtained from Aldrich Chemicals (St. Louis, Mo.). The acetate buffer was prepared from acetic acid, sodium acetate, and sodium chloride, all obtained from Aldrich Chemicals (St. Louis, Mo.).

### EXAMPLE 1

The production of hydroxyl radicals was detected by watching the increase in fluorescence intensity when terephthalic acid, a non-fluorescing species, was exposed to titanium dioxide.

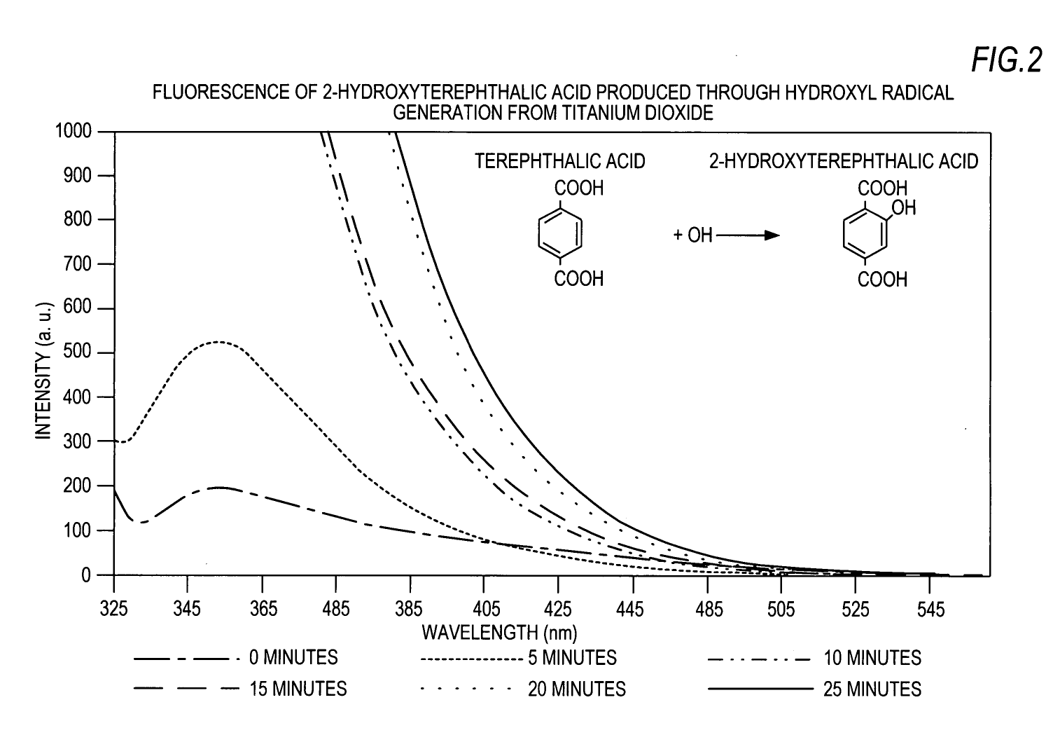

A clean glass slide was coated with titanium dioxide. A 9 mm×25 mm segment of the titanium dioxide coated slide was immersed in a cuvet containing 5×10⁻⁴ M terephthalic acid. A baseline fluorescence was taken at 0 minutes illumination. Fluorescence was then measured using a fluorimeter after 5 minute intervals of illumination. White light from a halogen bulb was used without any bandpass filters. As can be seen in FIG. 2, increased fluorescence intensity was detected, which indicated an increase in the production of 2-hydroxy terephthalic acid, a fluorescent species. As a control, the experiment was repeated using a glass slide omitting the titanium dioxide coating. The fluorescence intensity only slightly increased (<0.05 difference in intensity).

### EXAMPLE 2

A clean 3 cm×5 cm microscope slide was washed for one minute with ethanol and then rinsed with copious amounts of water. The slide was blast air-dried until all visible water was absent. Titanium dioxide was deposited on the slide by spin coat method. 1.0 mL of titanium tetraisopropanate was dropped onto the slide while spinning at 100 rpm on a standard spin coater. The slide was allowed to spin for ten seconds and then was removed. The slide was allowed to react with atmospheric water for about 10 to 30 minutes until it appeared coated with white powder. The slide was annealed for two hours at 400° C. in a vacuum oven. After cooling, the slides were rinsed with water and sonicated for two minutes each. Each slide was blast air-dried until all visible water was absent.

Polydimethylsiloxane (i.e., PDMS) was cured in a pitre dish without any channel molds present. 5 mm×25 mm channels were cut out of the PDMS and then laid on the titanium dioxide coated glass surface producing an open channel. A 3 mg/mL aqueous solution of myoglobin (horse skeletal) was prepared and placed in the PDMS channels. A handheld ultraviolet shortwave device was placed over the channels at 0.5 cm. The channels were illuminated for the specified period of time. The protein fragments solution was then pipetted out of the channel and diluted with 3 mM N-Tris(hydroxymethyl)methyl-3-aminopropanesulfonic acid in 10:1 (N-Tris(hydroxymethyl)methyl-3-aminopropanesulfonic acid/protein fragment solution ratio for capillary electrophoresis.

Capillary electrophoresis of the fragments was performed using a Beckman CE. The experimental parameters were run at +22 kV with 25 mM pH 4.5 acetate buffer as the running buffer. The buffer was adjusted to an ionic strength of 25 mM with NaCl. Samples were electrokinetically injected at 10 kV for 5 seconds. The capillary was 75 μm i.d. with a detection bubble of 200 μm. The effective length of the capillary was 37 cm. The capillary had previously been coated with poly (hydroxyethy)acrylic acid (i.e., PHEA) solution. Briefly, a 1.0 M solution of NaOH was flushed through the capillary at 5 psi for 15 minutes. Then a 1% w/v solution of PHEA was flushed through the capillary for 15 minutes at 5 psi. The capillary was rinsed 1 minute each with water and buffer solution initially and then before each use.

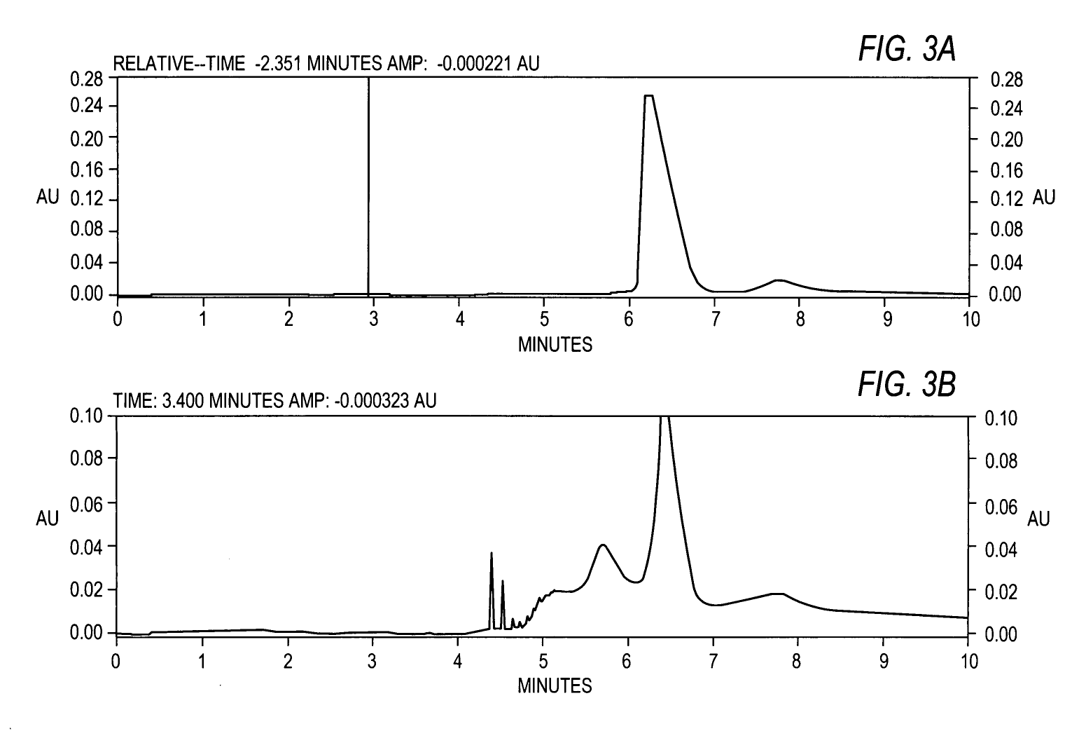

As shown in FIG. 3B, additional peaks evidencing a cleavage pattern of the myoglobin were seen when the myoglobin was exposed to illuminated titanium dioxide.

### EXAMPLE 3

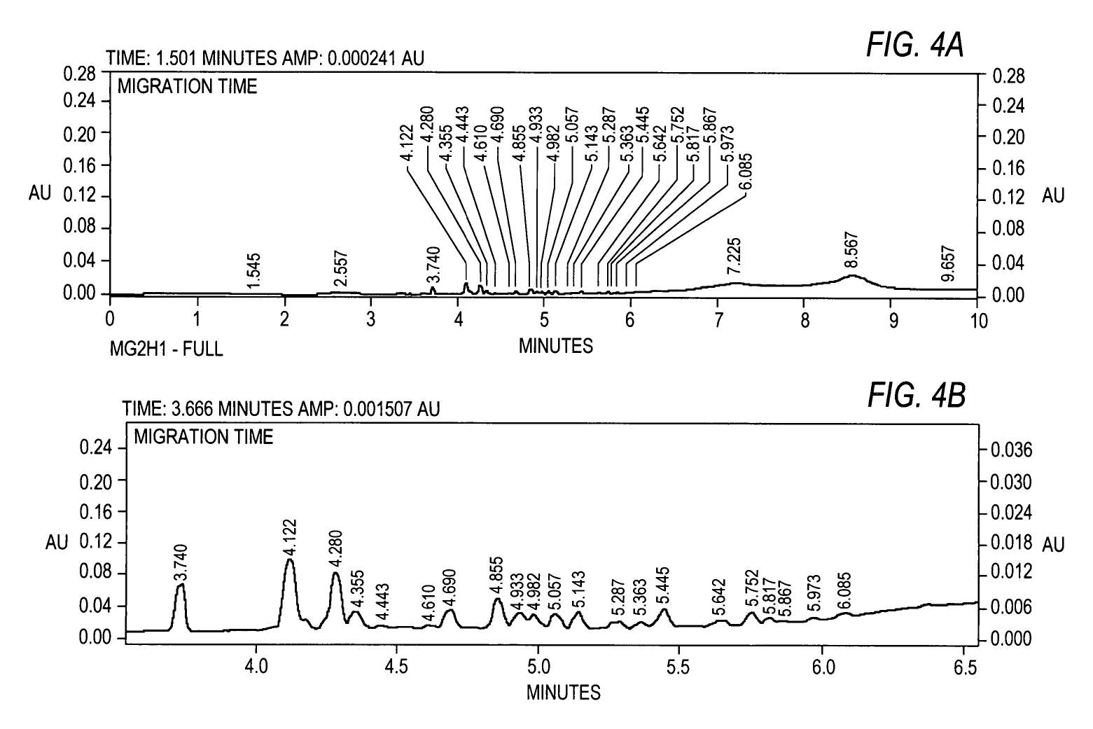

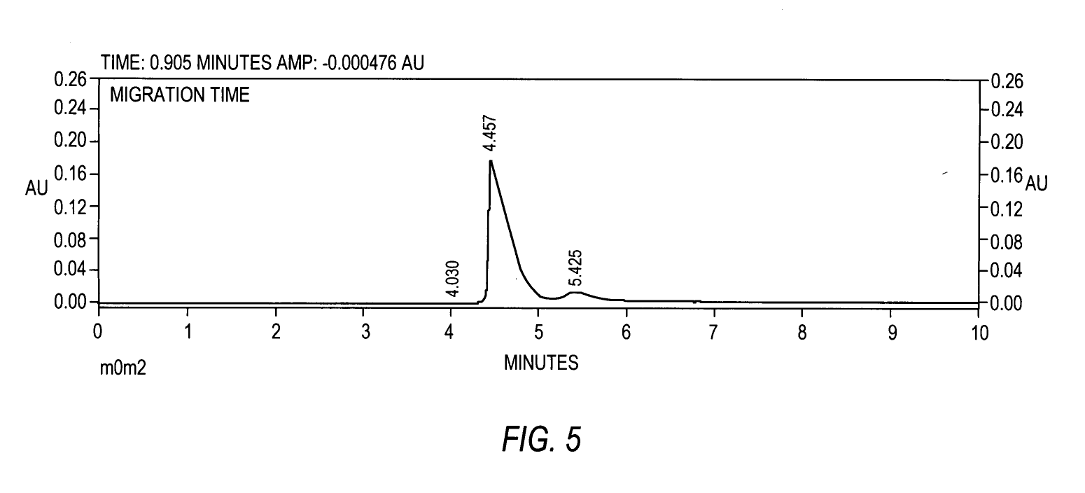

The experimental procedure in this example was the same as Example 2 except for the fact that air was incorporated into the sample to increase the oxygen content. As can be seen from FIGS. 4A-4B, when the myoglobin sample was illuminated for two hours, a greater amount of cleavage of the myoglobin occurred.

### EXAMPLE 4

A clean 3 cm×5 cm microscope slide was washed for one minute with ethanol and then rinsed with copious amounts of water. The slide was blast air-dried until all visible water was absent. Titanium dioxide was deposited on the slide by spin coat method. 1.0 mL of titanium tetraisopropanate was dropped onto the slide while spinning at 100 rpm on a standard spin coater. The slide was allowed to spin for ten seconds and then was removed. The slide was allowed to react with atmospheric water until it appeared coated with white powder. The slide was annealed for two hours at 400° C. in a vacuum oven. After cooling, the slides were rinsed with water and sonicated for two minutes each. Each slide was blast air-dried until all visible water was absent.

Polydimethylsiloxane (i.e., PDMS) was cured in a pitre dish without any channel molds present. 5 m×25 mm channels were cut out of the PDMS and then laid on the titanium dioxide coated glass surface producing an open channel. A second PDMS device was then fabricated. 5 mm diameter wells were cut from the cured PDMS using a cork bore, and then placed on the titanium dioxide coated slide.

A 3 mg/mL aqueous solution of myoglobin (horse skeletal) was prepared. 200 μL of the solution was placed in the open PDMS channels. Illumination of the myoglobin in the wells was performed using a Zeiss microscope with a bandpass filter at 380 nm. The 20× objective was used and the light was focused for illumination of the well. The wells were placed on the microscope platform at 2.5 centimeters from the objective. By pipetting the protein fragments solution into a 5 mm diameter well and illuminating it with a microscope having a bandpass filter for 380 nm, the volume to surface area ratio was controlled and the ability to quantify the light energy delivered to the microfluidic device was improved.

The protein fragments solution was then pipetted out of the channel and diluted with 3 mM N-Tris(hydroxymethyl)methyl-3-aminopropanesulfonic acid in 10:1 (N-Tris(hydroxymethyl)methyl-3-aminopropanesulfonic acid/protein fragment solution ratio for capillary electrophoresis. A total volume of 200 microliters was achieved. Capillary electrophoresis of the fragments was performed using a Beckman CE. The experimental parameters were run at +22 kV with 25 mM pH 4.5 acetate buffer as the running buffer. The ionic strength was not adjusted for the buffer. The native ionic strength was 8 mM. Samples were electrokinetically injected at 10 kV for 5 seconds. The capillary was 75 μm i.d. with a detection bubble of 200 μm. The effective length of the capillary was 37 cm.

The capillary had previously been coated with poly (hydroxyethy)acrylic acid (i.e., PHEA) solution. Briefly, a 1.0 M solution of NaOH was flushed through the capillary at 5 psi for 15 minutes. Then a 1% w/v solution of PHEA was flushed through the capillary for 15 minutes at 5 psi. The capillary was rinsed 1 minute each with water and buffer solution initially and then before each use.

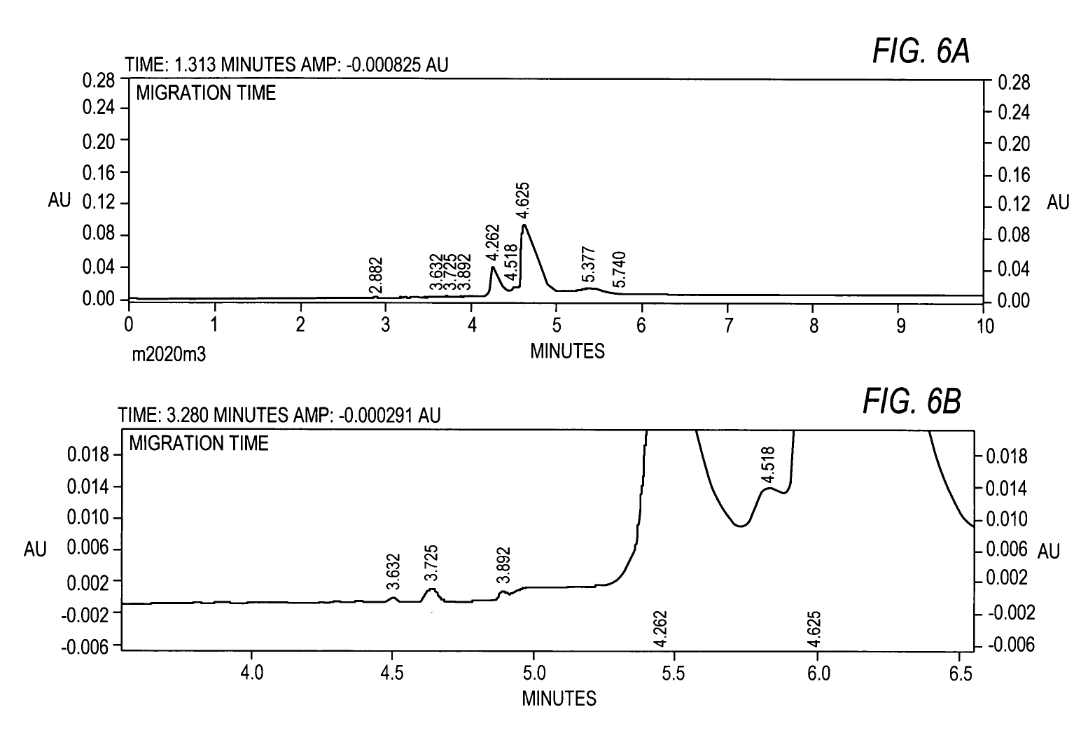

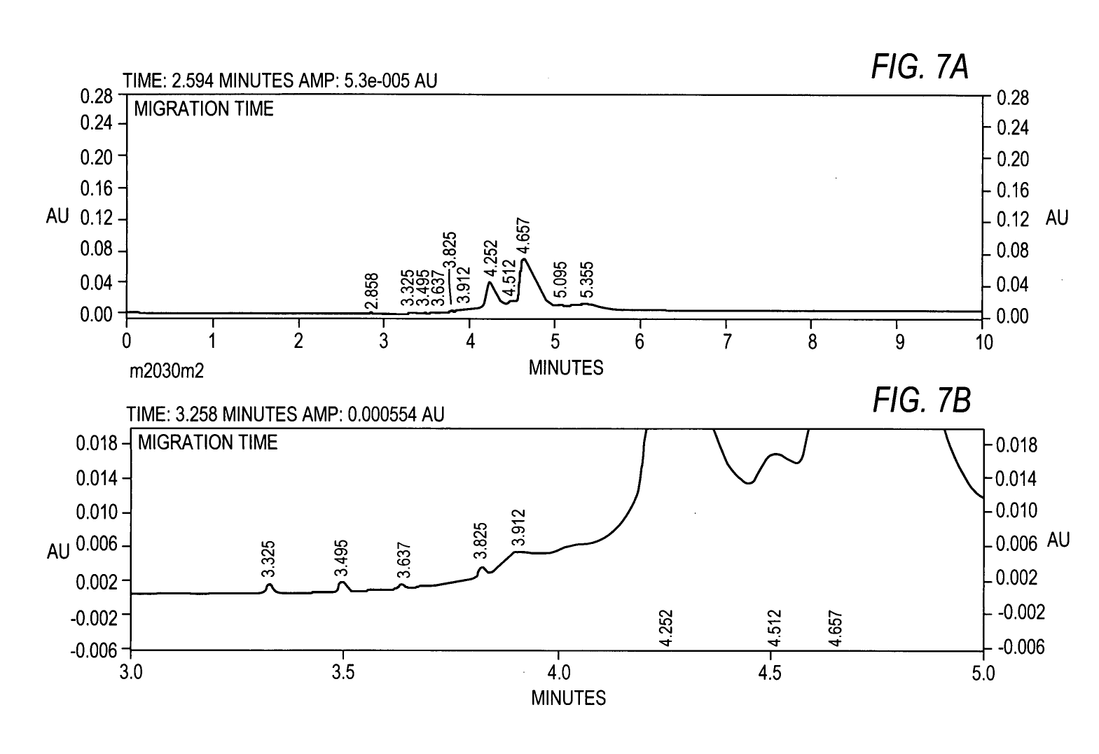

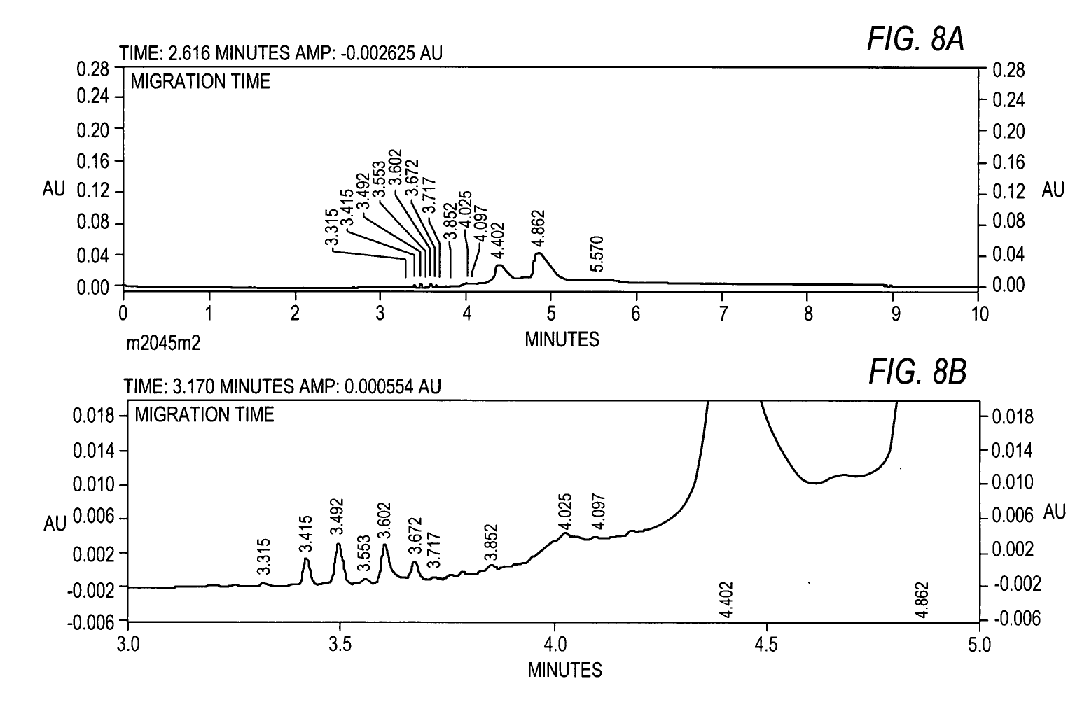

Proof of the successful cleavage of the myoglobin was found in FIGS. 6A-8B, which show that the protein was cleaved into distinct and reproducible parts by hydroxyl radicals generated from ultraviolet irradiation of titanium dioxide. Thus, the inventive method provides a method of cleaving biomolecular components in a biological sample with the use of hydroxyl radicals produced from excited semiconductors.

It will be apparent to those skilled in the art that various modifications and variations can be made without departing from the spirit or scope of the invention. Thus, it is intended that the present invention cover the modifications and variations of the invention, provided they come within the scope of the appended claims and their equivalents.

1. A microfluidic device for cleaving a biomolecular component in a biological sample, comprising:
a microchip having a plurality of channels, wherein at least one of the channels includes an inorganic semiconductor disposed on a portion of an interior surface of the channel; and
an energy source disposed at a position external to the channel for excitation of the inorganic semiconductor thereby generating hydroxyl radicals to effect cleavage of a biomolecular component in a biological sample.

2. The device of claim 1, wherein the portion of the channel including the inorganic semiconductor is in the form of a cleavage chamber.

3. The device of claim 1, further comprising a detector means to detect the presence of the cleaved biomolecular component.

4. The device of claim 1, wherein the inorganic semiconductor is selected from the group consisting of titanium dioxide, zinc oxide, and combinations thereof.

5. The device of claim 1, wherein the inorganic semiconductor is covalently bonded to the interior surface of the channel.

6. The device of claim 1, wherein the inorganic semiconductor is covalently bonded to a plurality of particles in the cleavage chamber.

7. The device of claim 1, wherein the channel omits an organic cleavage agent.

8. The device of claim 1, wherein the energy source is selected from the group consisting of light energy, thermal energy, electrical energy, and combinations thereof.

9. The device of claim 1, wherein the energy source is located on the microchip.

10. The device of claim 1, wherein the energy source is disposed at a position external to the microchip.

11. The device of claim 1, wherein the microchip is electronically coupled to a power source.

12. The device of claim 1, wherein the microchip further comprises a sample inlet well for introducing the biological sample, the sample inlet well being fluidly coupled to the plurality of channels.

13. The device of claim 1, wherein the microchip further comprises at least one separation channel located at a position intermediate the sample inlet well and the channel including the inorganic semiconductor.

14. The device of claim 1, wherein the microchip further comprises at least one outlet well for permitting waste to egress from the microchip, the at least one outlet well being fluidly coupled to the plurality of channels at a position distal from the sample inlet well.

15. The device of claim 1, further comprising at least one inlet well for introduction of an aqueous medium.

16. The device of claim 1, wherein the walls of the plurality of channels comprise fused silica, polydimethlysiloxane, polycarbonate, and combinations thereof.

17. The device of claim 1, wherein the biomolecular component is selected from the group consisting of amino acid sequences, nucleic acid sequences, polysaccharides, and combinations thereof.

18. A method of cleaving a biomolecular component in a biological sample in a microfluidic device, which comprises:
providing a microchip having a plurality of channels, wherein at least one of the channels includes an inorganic semiconductor disposed on a portion of an interior surface of the channel;
introducing a biological sample into the microchip;
inducing flow of the biological sample through the microchip; and
exciting the semiconductor thereby generating hydroxyl radicals to effect cleavage of a biomolecular component in the biological sample.

19. The method of claim 18, wherein the portion of the channel including the inorganic semiconductor is in the form of a cleavage chamber.

20. The device of claim 18, wherein the biomolecular component is selected from the group consisting of amino acid sequences, nucleic acid sequences, polysaccharides, and combinations thereof.

21. The method of claim 18, further comprising the step of detecting the presence of the cleaved biomolecular component.

22. The method of claim 18, wherein the channel omits an organic cleavage agent.

23. The method of claim 18, further comprising providing an aqueous medium in at least one inlet well, the aqueous medium comprising a buffering agent.

24. The method of claim 18, wherein detection is measured by either ultraviolet absorption or fluorescence.

25. The method of claim 18, wherein the flow of the biological sample is induced by an electric potential.

26. A method of cleaving a biomolecular component in a biological sample, which comprises:
providing a substrate, wherein at least one portion of the substrate includes an inorganic semiconductor for cleaving a biomolecular component in a biological sample;
introducing the biological sample on the substrate;
inducing flow of the biological sample over the substrate; and
exciting the semiconductor thereby generating hydroxyl radicals to effect cleavage of the biomolecular component.

27. The method of claim 26, wherein the portion of the substrate including the inorganic semiconductor is in the form of a cleavage chamber.

28. The device of claim 26, wherein the biomolecular component is selected from the group consisting of amino acid sequences, nucleic acid sequences, polysaccharides, and combinations thereof.

29. The method of claim 26, further comprising the step of detecting the presence of the cleaved biomolecular component.

30. The method of claim 26, wherein the substrate omits an organic cleavage agent.

31. The method of claim 26, further comprising providing an aqueous medium in at least one inlet well, the aqueous medium comprising a buffering agent.

32. The method of claim 26, wherein detection is measured by either ultraviolet absorption or fluorescence.

33. The method of claim 26, wherein the flow of the biological sample is induced by an electric potential.
